(19) United States
(12) Patent Application Publication
(10) Pub. No.: US 2026/0066068 A1
(43) Pub. Date: Mar. 5, 2026

(54) Information Processing Method, Information Processing System, and Computer Readable Medium

(71) Applicant: The University of Osaka, Suita-shi, Osaka (JP)

(72) Inventors: Kenji Yamada, Toyonaka-shi, Osaka (JP); Shogo Ochiai, Toyonaka-shi, Osaka (JP); Junya Kurahashi, Toyonaka-shi, Osaka (JP)

(21) Appl. No.: 19/104,933

(22) PCT Filed: Aug. 23, 2023
(86) PCT No.: PCT/JP2023/030231

§ 371 Date: Feb. 19, 2025

(30) Foreign Application Priority Data
Aug. 23, 2022 (JP) 2022-132625

(51) Int. Cl. G16H 10/60; G16H 30/20; H04L 9/00

(52) U.S. Cl. CPC G16H 10/60; G16H 30/20; H04L 9/50

(57) ABSTRACT

Provided are an information processing method, an information processing system, and a computer program enabling a data distribution while preventing falsification or fraud by using a non-fungible token (NFT). An information processing method includes processing of storing medical data relevant to a medical examination of a patient in a storage, minting an NFT corresponding to the stored medical data for a blockchain account of the patient in a blockchain system, and allowing an access to the medical data from a device of an owner of the NFT.

### CROSS-REFERENCE TO RELATED APPLICATIONS

This application is the national phase under 35 U.S.C. § 371 of PCT International Application No. PCT/JP2023/030231 which has an International filing date of Aug. 23, 2023 and designated the United States of America.

### FIELD

The present invention relates to an information processing method for medical data, an information processing system, and computer readable medium storing a computer program using a blockchain system.

### BACKGROUND ART

In accordance with an advance in a communication technology and a semiconductor technology, it has been possible to transmit and receive a vast amount of information on a network. However, there is a case where it is difficult to certify the authenticity of data to be transmitted and received, there is a concern that data is falsified, and there also is data not to be transmitted and received in consideration of high duplicability of digital data.

For example, recently, various remote medical examination systems enabling a medical doctor in a remote region to perform a medical examination on a patient have been continuously proposed, but data is individual information, and it is necessary to transmit and receive medical data according to human life, and thus, it is difficult to attain such systems in a closed network. In a case where measurement data is for the same person, but there is no coordination between measurement devices or measurement is performed in different facilities, it is difficult to handle the data in a cross-sectional manner even when it is information for the same person.

Regarding data security such as falsification, various systems using a data distribution using a blockchain are proposed.

In proposed system, buying and selling access right for IoT data as a token is disclosed.

### SUMMARY

A system enabling a cross-sectional access from a plurality of medical institutes or researchers while avoiding the management of the intensive storage of data to be confidential, such as medical data, in a server of a client server system is expected.

The present invention has been made in consideration of such circumstances, and an object thereof is to provide an information processing method, an information processing system, and a computer program enabling a data distribution while preventing falsification or fraud by using a non-fungible token (NFT).

In an information processing method of one embodiment of the present disclosure, medical data relevant to a medical examination of a patient is stored in a storage, an NFT corresponding to the stored medical data is minted for a blockchain account of the patient in a blockchain system, and an access to the medical data from a device of an owner of the NFT is allowed.

In the information processing method of the present disclosure, the NFT based on the medical data relevant to the medical examination of the patient is minted for the patient. The medical data may be measurement data generated by a first device that is used by the patient or a medical device that is connected to the first device, or may be a chart (a medical chart) in a medical institute in which the patient is subjected to the medical examination. The medical data may be stored in the storage from a second device corresponding to the medical institute, and a request for minting the NFT may be transmitted from the second device. The access to the medical data on the storage may be allowed from the medical institute of a medical examination destination of the patient. The access to the medical data is not allowed to others except for the medical institute unless owning the NFT.

In the information processing method of the present disclosure, the storage may be a distributed storage, or may be a storage that is accessed via a server.

The stored data may be medical data according to various medical diagnoses for one patient, or may be data relevant to a growing process of agricultural crops.

According to the present disclosure, the access to the data stored in the storage accessible from a plurality of devices can be controlled by using the NFT. By using the NFT, it is possible to transfer the data while making it difficult to falsify data (uniform resource identifier: URI) for accessing the data and the owner. Accordingly, it is possible to increase the utility value of the data while making it possible to share the data.

The above and further objects and features will more fully be apparent from the following detailed description with accompanying drawings.

### BRIEF DESCRIPTION OF DRAWINGS

FIG. 1 is a schematic view of a remote medical care system of this embodiment.

FIG. 2 is a block diagram illustrating a configuration of a first device.

FIG. 3 is a block diagram illustrating a configuration of a second device.

FIG. 4 is a block diagram illustrating a configuration of a third device.

FIG. 5 is a block diagram illustrating a configuration of a node in a blockchain system.

FIG. 6 is a sequence diagram illustrating a processing procedure executed by the remote medical care system.

FIG. 7 is a diagram illustrating a screen example displayed on the first device.

FIG. 8 is a diagram illustrating a screen example displayed on the first device.

FIG. 9 is a sequence diagram illustrating a transaction processing of an NFT of medical data.

FIG. 10 illustrates a screen example displayed on the third device.

FIG. 11 illustrates a content example of a detailed screen.

FIG. 12 illustrates a notification screen with respect to the first device.

FIG. 13 illustrates a search screen when minting a plurality of NFTs.

FIG. 14 is a sequence diagram illustrating a procedure of burning processing of the NFT.

### DESCRIPTION

The present disclosure will be described in detail, with reference to the drawings illustrating the embodiment of the present disclosure. In the following embodiment, a remote medical examination system to which an information processing method of the present disclosure is applied will be described.

In the present disclosure, a “blockchain system” indicates a system that includes a plurality of computers connected to each other such that communication is available, and a network connecting the plurality of computers, and creates a blockchain by distributed processing of the plurality of computers. In the blockchain, the contents of a transaction to be required are imported.

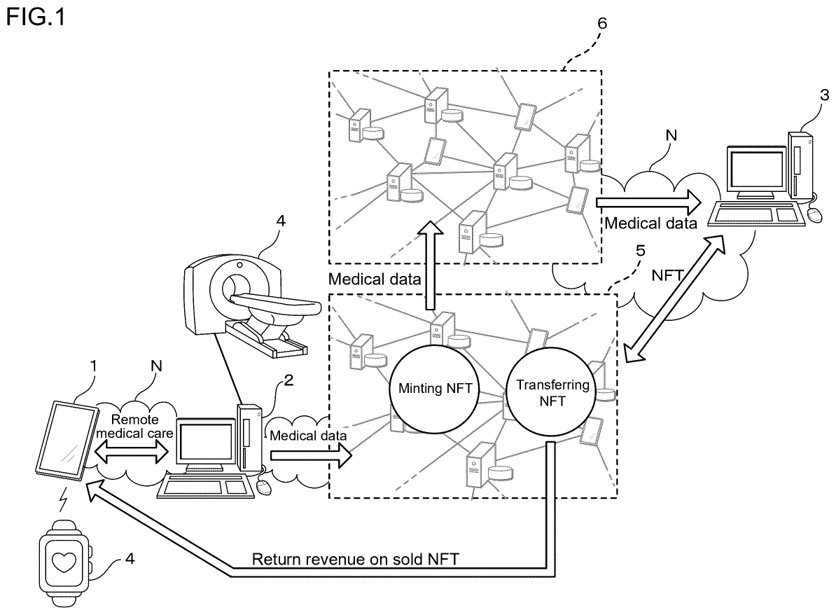

FIG. 1 is a schematic view of a remote medical examination system 100 of this embodiment. The remote medical examination system 100 includes a first device 1 used by a user who is a patient, a second device 2 used by a medical institute, a third device 3 used by another user, a blockchain system 5, and a distributed storage 6. The remote medical examination system 100 includes a network N transmitting and receiving data between the first device 1, the second device 2, the third device 3, the blockchain system 5, and the distributed storage 6.

In the blockchain system 5, for example, a blockchain based on a protocol referred to as a secret network is used. The blockchain system 5 may be a blockchain system based on Ethereum (Registered Trademark). It is preferable that the blockchain system 5 is a public chain, and a blockchain using a protocol that can be used in a smart contract executing the following processing.

The first device 1, the second device 2, or the third device 3 is capable of sending a request to the smart contract on the blockchain system 5 that is the public chain. In the blockchain system 5, the smart contract minting a non-fungible token (NFT) on the basis of data relevant to a medical examination (hereinafter, referred to as medical data) is deployed. The blockchain system 5 is not limited to the secret network-based blockchain system or the Ethereum-based blockchain system, and may be a blockchain system based on other blockchain standards capable of minting the NFT.

In the distributed storage 6, for example, a Dfinity-based blockchain is used. The blockchain applied to the distributed storage 6 is a public chain, and is accessible from any of the first device 1, the second device 2, and the third device 3. Specifically, the distributed storage 6 is configured by including a plurality of distributed storage media. In a case where the medical data is written in the distributed storage 6, an address (for example, URI or identification data) for accessing the medical data is obtained, and the stored medical data is accessible from the address. The distributed storage 6, for example, may be storage based on an inter planetary file system (IPFS). In a case where processing of adding the medical data to the distributed storage 6 is executed even when using the IPFS, an address (identification data) for accessing the medical data is obtained, and the medical data is accessible from the address.

The distributed storage 6 is not limited to the Dfinity-based storage or the IPFS-based storage, and may be in the form of a distribution server that is not a blockchain. In this case, at least the hash value of the data and the URI of the data are imported in the block of the blockchain system 5 such that whether the data is falsified can be verified. It is preferable that the distributed storage 6 is configured as a decentralized system that is less likely to be falsified while ensuring attack resistance. Therefore, it is preferable that the distributed storage 6 is stored in a blockchain in which image data, moving image data, and the like, having a comparatively large data size, can also be stored. Further, the distributed storage 6 may be the same as the blockchain system 5. When the medical data is stored in the distributed storage 6 that is not a blockchain, it may be configured such that the medical data can be automatically and securely stored in the first device 1 or the second device 2, on the basis of electronic contract information.

The network N is a communication network including a carrier network and a public communication network (the internet). The network N may include a dedicated line. The carrier network includes a base station. The public communication network includes an access point. For example, the first device 1 is capable of communicating with the second device 2 via the base station and the network N. The first device 1, the second device 2, and the third device 3 are capable of transferring data such as the medical data between the blockchain system 5 and the distributed storage 6 via the network N.

In the remote medical examination system 100, the user is a patient in the medical care. The remote medical examination system 100 sequentially stores storage relevant to the medical care of the user as the medical data in the distributed storage 6. For the storage, the remote medical examination system 100 mints an NFT for which the user is originally user set as an owner in the blockchain system 5. Further, the remote medical examination system 100 controls the use of the medical data stored in the distributed storage 6 by using the ownership of the NFT.

In the remote medical examination system 100, the medical data is not stored in a confidential storage in each medical institute, but is retained in the distributed storage 6 that is not accessible when falsified. Therefore, the medical data can be accurately browsed from each of a plurality of medical institutes of a medical examination destination of the user. When the patient who is diagnosed in a face-to-face manner in a certain medical institute is remotely diagnosed in another medical institute diagnosis, it is possible to refer to an examination result in the first medical institute on the basis of individual identification data of the patient. Accordingly, it is possible to expect that the reliability and the accuracy of remote medical diagnosis are maintained.

Further, in the remote medical examination system 100 of the present disclosure, by the following processing using the NFT and the distributed storage 6, it is possible for the third device 3 of a person who purchases the NFT to use the medical data of the user. That is, it is possible for a medical researcher, for example, who purchases the NFT, to use the medical data as valuable data in the third device 3. Further, by the remote medical examination system 100 using the NFT, it is also possible to return revenue according to the transfer of the NFT to the user who mints the NFT for the medical data. Accordingly, it is possible to use the medical data that is not falsified and is reliable in the remote medical care, and perform diagnosis and research on the basis of diversified diagnosis data of one patient.

A specific configuration for attaining such a remote medical examination system 100 will be described below.

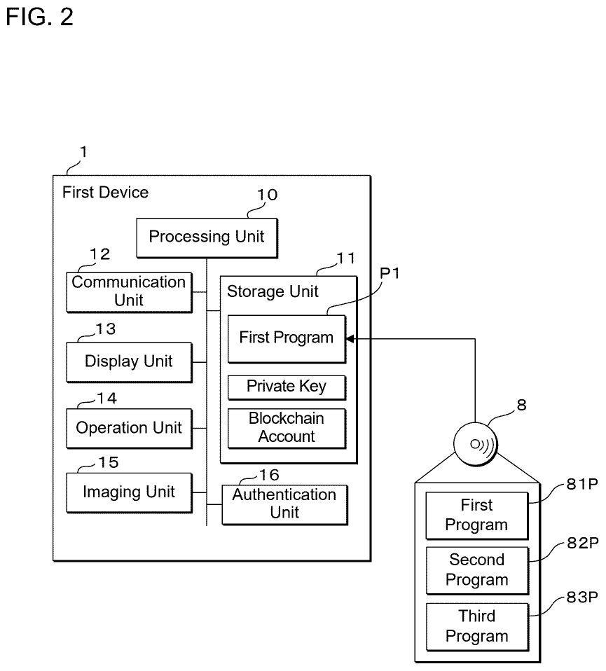

FIG. 2 is a block diagram illustrating the configuration of the first device 1. The first device 1 is, for example, a smartphone or a tablet terminal used by the patient. The first device 1 is not limited to the smartphone or the tablet terminal, and may be a personal computer in which communication is available. The first device 1 includes a processing unit 10, a storage unit 11, a communication unit 12, a display unit 13, an operation unit 14, an imaging unit 15, and an authentication unit 16.

In the processing unit 10, a processor such as a central processing unit (CPU) and a graphics processing unit (GPU), a memory such as a read only memory (ROM) and a random access memory (RAM), and the like are used. In the ROM, intrinsic identification data of the first device 1 is stored. The intrinsic identification data, for example, is a MAC address. The processing unit 10 may be configured as one piece of hardware (system on a chip: SoC) in which a processor, a memory, and the storage unit 11 and the communication unit 12 are integrated.

In the storage unit 11, a flash memory or a solid state drive (SSD) is used, and a program such as a first program P1 and data, which are referred to by the processing unit 10, are stored. The first program P1 is a program for causing the computer to function as the first device 1 of the remote medical examination system 100 of the present disclosure. In the storage unit 11, personal identity verification data for authenticating the user who is the patient is registered in advance. In the storage unit 11, the individual identification data of the user is stored. The individual identification data, for example, may be combined with a face photo and My Number (Registered Trademark: a personal identification number) of the user. As the individual identification data of the user, identification data of a health insurance card, identification data in the local government, and the like may be used. In the storage unit 11, as illustrated in FIG. 2, a private key of the user in the blockchain system 5 is stored. In this case, the private key may be stored in the ROM of the processing unit 10 or the storage unit 11 in a non-rewritable manner.

As the first program P1 stored in the storage unit 11, a first program 81P stored in a storage medium 8 readable from the computer may be read out by the processing unit 10 and stored in the storage unit 11.

The communication unit 12 is a communication module attaining communication connection with a communication device such as the second device 2, the blockchain system 5, or the distributed storage 6. In the communication unit 12, a network card, a wireless communication device, or a carrier communication module is used. The communication unit 12 may attain communication connection with the measurement device 4 that is a medical device. An interface for the communication connection with the measurement device 4 may be separately provided.

In the display unit 13, a display device such as a liquid crystal panel or organic EL display is used. The operation unit 14 is an interface receiving the operation of the user, and a physical button, a touch panel device with a built-in display, a speaker, a microphone, and the like are used as the operation unit 14. The operation unit 14 may accept the operation on a screen displayed on the display unit 13 by the physical button or the touch panel, may cognize operation contents from an input sound by the microphone, or may accept the operation in the format of a dialogue with the sound output by the speaker.

The imaging unit 15 is a camera imaging the patient using the first device 1. The imaging unit 15 is used to transmit and receive the own moving image of the user using the remote medical examination system 100 and make a call to a medical doctor or staff in the medical institute. The processing unit 10 is capable of acquiring an image signal (the moving image) imaged by the imaging unit 15 and transmitting the image signal toward a medical care provider via the communication unit 12.

The authentication unit 16 is a device for determining whether the patient using the first device 1 is coincident with the user registered in advance. The authentication unit 16 may be composed of a camera used in the imaging unit 15, and an arithmetic unit performing arithmetic of cognizing a face on an image obtained from the camera and arithmetic of deriving feature data of the face. The authentication unit 16 may be composed of a sensor for biological authentication, and an arithmetic unit deriving biological feature data from data obtained from the sensor. The processing unit 10 determines whether the feature data of the face or the biological feature data obtained from the authentication unit 16 is coincident with the personal identity verification data registered in advance in the storage unit 11 to perform authentication.

The first device 1 is connected to the measurement device 4 that is the medical device, and thus, is capable of importing the data measured by the measurement device 4. The measurement device 4 is capable of measuring vitals or a change in the body of the user. The measurement device 4 is a thermometer, an electrocardiographic monitor, a blood pressure monitor, a dynamometer, a pulse oximeter, and the like. The first device 1, for example, is capable of importing vital data such as a heart rate from the measurement device 4. The first device 1 may be connected to the measurement device 4 via the communication unit 12, or may be connected to the measurement device 4 via a separate input/output interface such as a universal serial bus (USB) or another bus. The processing unit 10 of the first device 1 transmits the data obtained from the measurement device 4 to the blockchain system 5 and the distributed storage 6, on the basis of the first program P1. The processing unit 10 is capable of acquiring the type of measurement device 4 and/or a measurement device ID that is identifiable for each measurement device 4, together with numerical information indicating vital data or a measurement result of the patient, from the measurement device 4.

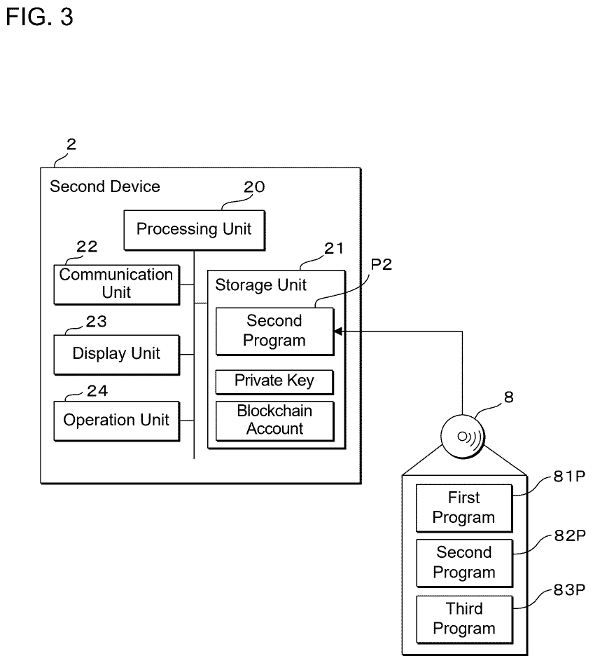

FIG. 3 is a block diagram illustrating the configuration of the second device 2. The second device 2 includes a processing unit 20, a storage unit 21, a communication unit 22, a display unit 23, and an operation unit 24. The hardware configuration of the second device 2 is the same as that of the first device 1, and thus, the corresponding reference numerals will be applied to omit the detailed description. The second device 2 has the function of a terminal in a chart (a medical chart) management system used by the medical care provider, in particular, the medical doctor. The second device 2 may be a smartphone or a tablet terminal as with the first device 1, and is preferably a desktop or laptop personal computer capable of executing image display processing on the brilliant display unit 23. The second device 2 is capable of communicating with the measurement device 4, a server included in the chart management system, or the like.

The storage unit 21 of the second device 2 stores a second program P2 for causing a general-purpose computer to function as the second device 2. As the second program P2, a second program 82P stored in the storage medium 8 readable from the computer may be read out by the processing unit 20 and stored in the storage unit 21. As the second program P2, a second program may be downloaded from an external distribution server and stored in the storage unit 21.

The second device 2 is also capable of importing data measured by the measurement device 4 in the medical institute. The processing unit 20 of the second device 2 is capable of acquiring the measurement device ID of the measurement device 4, the identification data (authentication data) of the patient measured by the measurement device 4, and the measured data via a network in the medical institute.

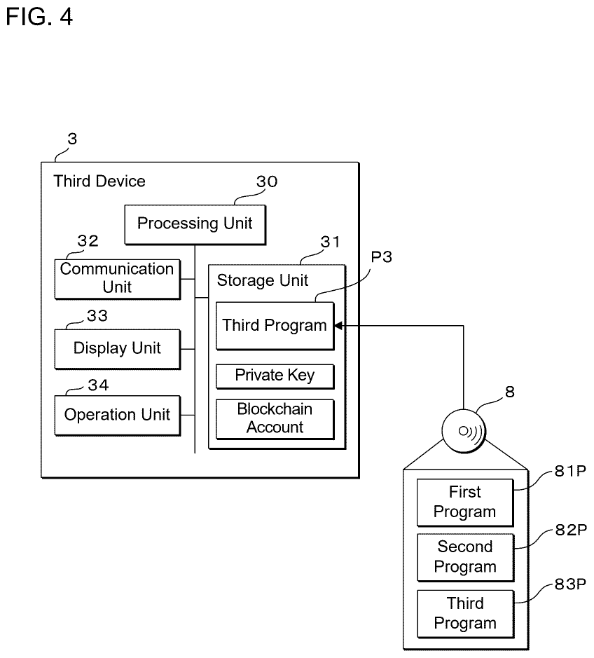

FIG. 4 is a block diagram illustrating the configuration of the third device 3. The hardware configuration of the third device 3 is the same as that of the first device 1 or the second device 2, and thus, the corresponding reference numerals will be applied to omit the detailed description. The third device 3, for example, is a desktop or laptop personal computer, or a server computer used by the researcher performing research using the medical data. The processing unit 30 is capable of searching for or receiving the medical data from the blockchain system 5 and the distributed storage 6, on the basis of a third program P3 stored in the storage unit 31.

The storage unit 31 of the third device 3 stores the third program P3 for causing a general-purpose computer to function as the third device 3. As the third program P3, a third program 83P stored in the storage medium 8 readable from the computer may be read out by the processing unit 30 and stored in the storage unit 31. As the third program P3, a third program may be downloaded from an external distribution server and stored in the storage unit 31.

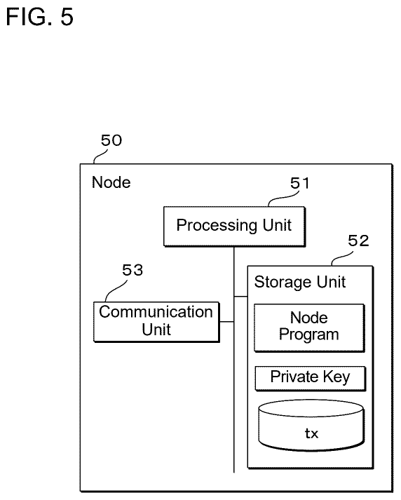

FIG. 5 is a block diagram illustrating the configuration of a node 50 in the blockchain system 5. The node 50 may be a server computer, may be a desktop or laptop personal computer, or may be a communication terminal device such as a smartphone. The node 50 includes a processing unit 51, a storage unit 52, and a communication unit 53. In addition, insofar as the node 50 is a device including at least the processing unit 51 and the communication unit 53, a part or the entirety of the nodes can be configured by a part of the processing unit 51.

In the processing unit 51, a processor such as a CPU and a GPU, a memory, and the like are used. The processing unit 51 may be configured as one piece of hardware in which a processor, a memory, and the storage unit 52 and the communication unit 53 are integrated. In the memory of the processing unit 51, a private key uniquely owned by each node 50 may be stored. Then, the processing unit 51 executes each piece of processing based on a node program stored in the storage unit 52, and causes a general-purpose computer to function as the node in the blockchain system 5.

In the storage unit 52, a hard disk or a flash memory is used, and a program such as the node program and data, which are referred to by the processing unit 51, are stored. The storage unit 52 stores a blockchain. In the node program, for example, a program for functioning as the smart contract minting the NFT. The private key described above may be stored in the storage unit 52. The storage unit 52 may store a public key and an address based on the private key.

The communication unit 53 is a communication module attaining intercommunication between the nodes 50. In the communication unit 53, a network card, an optical communication device, a wireless communication device, or the like is used.

On the basis of arithmetic in each of the nodes 50, various smart contracts described below are called, and defined processing is executed.

A processing procedure in the remote medical examination system 100 configured as described above will be described. In the remote medical examination system 100, for each data demander such as the user who is the patient, the medical care provider, and the researcher, it is necessary to set a wallet required for an access to at least the blockchain system 5 and/or the distributed storage 6. The setting of the wallet is the setting of a blockchain account. The blockchain account is minted to the user who is the patient, in association with the individual identification data of the user. The individual identification data of the user, for example, is a combination between the face photo and My Number (Registered Trademark) of the user stored in the first device 1. In the individual identification data of the user, the identification data of the health insurance card, the identification data in the local government, and the like may be used. Similarly, the blockchain account is minted to the medical care provider and the data demander. The blockchain account and a private key corresponding thereto are stored in the storage units 11, 21, and 31 of the first device 1, the second device 2, and the third device 3, respectively.

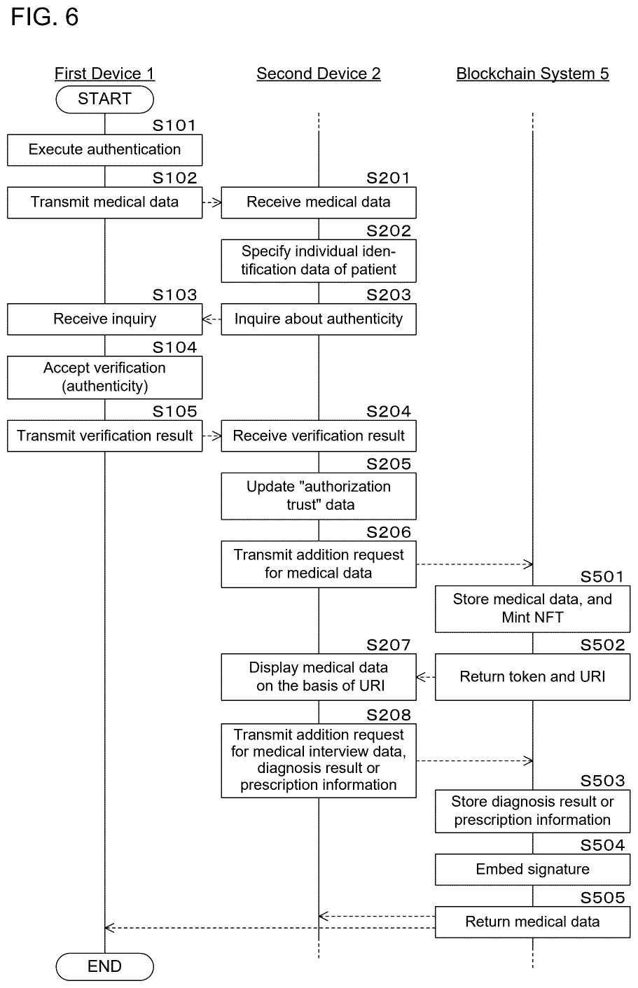

In a case where the wallet is set, by processing based on the first program P1 of the first device 1 and the second program P2 of the second device 2, as described below, the medical data of the patient is stored, and the NFT is minted. FIG. 6 is a sequence diagram illustrating the processing procedure executed by the remote medical examination system 100. The processing procedure illustrated in FIG. 6, for example, is started by the user or an assistant activating the first program P1 of the first device 1 to receive the medical examination using the remote medical examination system 100.

The processing unit 10 of the first device 1 executes authentication of whether an operator of the first device 1 or the patient in front of the first device 1 is user personal identity that is identified as the individual identification data by using the authentication unit 16 (step S101). The authentication in step S101 may be executed in cooperation with an authentication server inside or outside the remote medical examination system 100. In a case where the authentication in step S101 fails, the processing unit 10 displays an error on the display unit 13 without executing the subsequent sequence, and ends the processing. In a case where the authentication succeeds, the processing unit 10 continues the following sequence.

The processing unit 10 transmits the medical data (a set of a plurality of pieces of data according to the medical examination of the patient) including the data obtained from the measurement device 4 toward the medical institute of the medical examination destination corresponding to the user who is the patient (step S102). The medical institute of the medical examination destination may be registered in advance, or may be selected by the operation of the user before step S102. In step S102, the moving image data imaged by the imaging unit 15 to make a video call may be continuously imaged and transmitted afterward.

The processing unit 10 of the first device 1 may accept agreement on whether target medical data is provided to a data market in the distributed storage 6 from the user, before transmitting the medical data in step S102. The agreement may be performed each time when the medical data is transmitted, or the processing unit 10 of the first device 1 may collectively accept whether the medical data is used in the data market in the case of setting the wallet when using the remote medical examination system 100. The agreement on providing the medical data to the data market may be obtained by the staff such as the medical doctor in the medical care provider operating the second device 2 in the medical institute.

The medical data transmitted in step S102 may include at least the individual identification data of the user that is authenticated in step S101, blockchain account data, and the image data imaged by the imaging unit 15. The medical data may include the intrinsic identification data of the first device 1. The image data obtained from the imaging unit 15 includes identification data of the camera of the imaging unit 15, and “generation trust” data indicating that the image is extremely less likely to be falsified. The “generation trust” is data (a code) that can be verified when falsified. In a case where the measurement device 4 is connected to the first device 1, the medical data may include data obtained from one or a plurality of measurement devices 4. The data obtained from the measurement device 4, as described above, may include the measurement device ID (such as the MAC address) of the measurement device 4 and the data itself. The medical data includes the “generation trust” data indicating the data of the measurement device 4 is extremely less likely to be falsified at a time point when data is generated by each of the imaging unit 15 or the measurement device 4. The “generation trust” data, for example, is the hash value of the image data imaged by the camera, and the hash value of the data obtained from the measurement device 4.

The processing unit 10 of the first device 1 may accept the agreement on whether the target medical data is provided to the data market in the distributed storage 6 from the user before transmitting the medical data in step S102. The agreement may be performed each time when the medical data is transmitted, or the processing unit 10 of the first device 1 may collectively accept whether the medical data is used in the data market in the case of setting the wallet when using the remote medical examination system 100. The agreement on providing the medical data to the data market may be obtained by the staff such as the medical doctor in the medical care provider operating the second device 2 in the medical institute. In a case where the agreement is obtained, the medical data may include data indicating that the agreement is obtained.

In the medical institute of the medical examination destination, in a case where the second device 2 receives the medical data from the first device 1 of the patient (step S201), the processing unit 20 of the second device 2 specifies the individual identification data of the user who is the patient included in the received medical data (step S202). The processing unit 20 of the second device 2 inquires of the first device 1 about the authenticity of the transmitted medical data (step S203).

The processing unit 10 of the first device 1 receives the inquiry (step S103), and accepts verification of whether the medical data transmitted in step S102 is the own data (the authenticity) from the user on the screen displayed on the display unit 13 (step S104). In a case where the verification is accepted, the processing unit 10 transmits a verification result to the second device 2 (step S105).

The second device 2 receives the verification result (step S204), and updates “authorization trust” data indicating that authorization for the medical data (the determination of the authenticity) is obtained (step S205). The “authorization trust” is data indicating a result of the determination of the authenticity (such as a binary value of TRUE or FALSE). In a case where in step S103, the verification is not attained by the first device 1 due to the own cognition problem of the patient or difficulty in the operation, the “authorization trust” may be updated on the basis of the operation with respect to the second device 2 by the medical care provider. In this case, as the “authorization trust”, for example, cognition level and disability level data signed by the blockchain account of the second device 2 of the medical care provider is added to the medical data. The processing unit 20 transmits an addition request for the medical data to the blockchain system 5 (step S206). The addition request includes a wallet address (the blockchain account) relevant to the individual identification data of the patient.

In the blockchain system 5, the medical data is stored and the NFT is minted for the medical data by the smart contract that is called by the addition request for the medical data (step S501). The blockchain system 5 verifies and approves the transmitted transaction by the blockchain account associated with the individual identification data of the user who is the owner of the medical data.

In step S501, the smart contract of the blockchain system 5 stores the medical data such as an image in a JSON format in the distributed storage 6, and specifies the URI thereof. The medical data stored in the distributed storage 6 is encoded. The transaction stored by the smart contract of the distributed storage 6 may be accepted and executed. The smart contract of the blockchain system 5 mints the NFT for the medical data specified by the specified URI for the user (the patient). The minted NFT includes a token ID, the individual identification data of the patient, and the URI of the medical data in the distributed storage 6, and is imported in the block of the blockchain system 5.

The smart contract of the blockchain system 5 returns the token ID of the minted NFT, and the stored URI to the second device 2 (step S502).

The second device 2, on the basis of the returned URI, displays the medical data of the patient stored as metadata relevant to the minted NFT as the chart (the medical chart) on the display unit 23 (step S207). In the second device 2, the medical data that is encoded and stored in the distributed storage 6 can be decoded and displayed, on the basis of the second program P2. Here, the medical data includes not only the medical data of a target patient stored in step S501, but also a measurement result, medical interview data, medication information, genetic information, and the like of the target patient in the past.

In a case where the chart displayed on the second device 2 is checked by the medical doctor, and a text of medical interview data, a diagnosis result, or prescription information is added, the processing unit 20 transmits an addition request for also adding the added data as the medical data of the same user to the blockchain system 5 (step S208).

The smart contract of the blockchain system 5 adds the transmitted medical interview result, diagnosis result, and prescription information to the medical data of the patient (step 503). The smart contract embeds a signature in a public key cryptosystem in the medical data (step S504), and returns the medical data to the first device 1 or the second device 2 (step S505). Accordingly, the medical data authorized by the medical doctor by consent of the patient is stored such that the medical data is difficult to be falsified, and processing of one medical examination is ended.

In the processing of step S207 and steps S503 to S505, the data may be added to the metadata corresponding to the token with the same ID that can be specified by the same URI, or another token may be minted and executed, and may be stored dividedly in a plurality of URIs each time when information is added to the individual identification data of the same user (an event occurs).

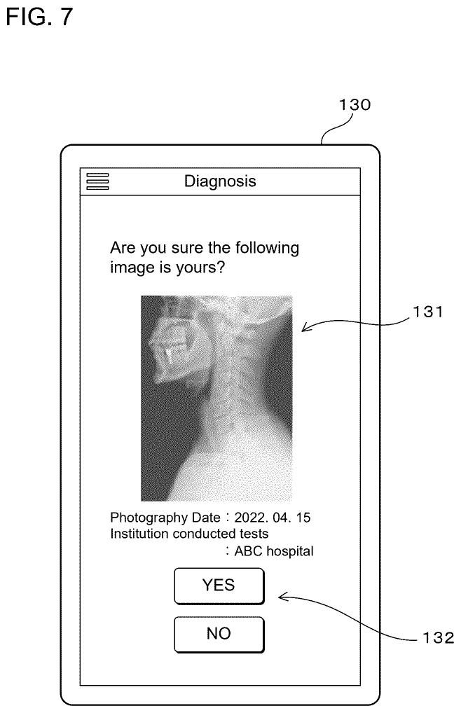

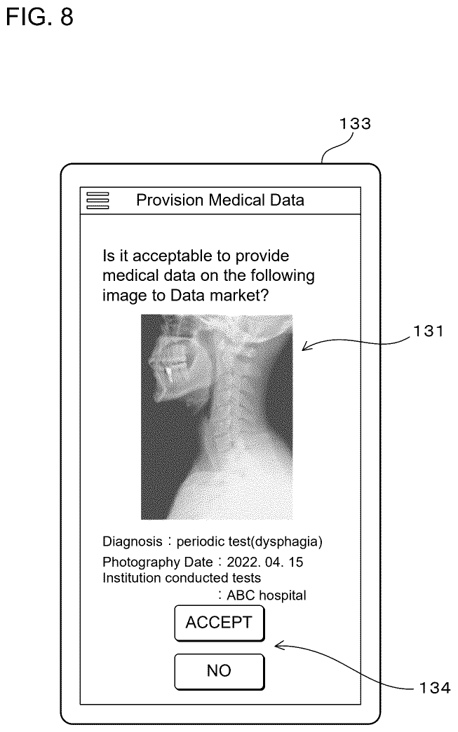

FIG. 7 and FIG. 8 are diagrams illustrating a screen example displayed on the first device 1. FIG. 7 illustrates a screen example displayed in step S103. In a screen 130 used by the user who is the patient, image data 131 included in the medical data, and an icon 132 for accepting a response for the authenticity are included. In a case where the user or the assistant selects the icon 132, the result of the authenticity is transmitted to the second device 2.

FIG. 8 illustrates a screen example displayed before step S102. In a screen 133 of FIG. 8, among data measured for the patient using the first device 1, the image data 131 is displayed, as with FIG. 7. In the screen 133, an icon 134 for accepting whether to provide the medical data to the data market in the distributed storage 6 is displayed. In a case where the user selects the icon 134, the authorization by the patient is transmitted to the second device 2.

In this embodiment, during the sequence illustrated in FIG. 6 or retroactively, data referred to as “cognition trust” (included in the medical data) indicating whether the patient has cognitive ability may be updated. The “cognition trust”, for example, is data including a numerical value indicating a dementia level and a numerical value indicting a daily life independence level. The “cognition trust” may be data that is added by the second device 2 on the authority of the medical doctor. By the “cognition trust”, it is possible to objectively determine whether the data stored in the distributed storage 6 can be shared by others.

As described above, in the distributed storage 6, the encoded medical data is stored in a public blockchain. For the same patient, the medical data is stored each time when measurement is performed by the measurement device 4 that is the medical device and each time when a remote diagnosis is performed in the medical institute. Not only in a case where a remote medical examination is performed between the first device 1 and the second device 2, but also in a case where the measurement data such as X-ray or MRI is directly obtained by the medical device in the second device 2, the processing of step S201 to step S207 and step S501 to step S505 is executed. The processing illustrated in the sequence diagram of FIG. 6 is also executed in a case where the processing is performed on the same patient in different medical institutions. The medical data of the same patient is stored by using the blockchain account associated with the individual identification data of the patient such as My Number, and thus, can be treated as data of the same patient even when stored via different medical institutes. Insofar as it is the medical data of the same patient, the same patient is the first owner of the NFT of such medical data.

The medical data stored in the distributed storage 6, for example, includes any of medical examination memory (the moving image data in the remote medical care), transportation memory, the identification data of the medical institute in which the medical examination is performed, medical examination memory by the medical doctor, the prescription information, medical interview data (questionnaire data such as medication, adverse effects, and allergies verified in advance), the measurement data by the measurement device 4, and the like. The measurement data, as described above, may include not only data of a heart rate meter, a blood pressure monitor, or the like that can be remotely measured on the user side, but also any of examination results (such as an X-ray image and an MRI image) by the medical device in the medical institute, and the like. In addition, the medical data may include hospitalization memory, nursing-related information (ADL and the level of care needed), the medication information, basic physical information (such as a body composition and a foot size), the evaluation of a cognition function, the evaluation of a deglutition function, and the evaluation of a walking function. The medical data includes data indicating that the patient agrees with being the NFT.

The medical data stored in the distributed storage 6 can be used (downloaded) by the owner of the corresponding NFT, in addition to the second device 2 used by the medical doctor of the diagnosis destination of the patient of the medical data. Next, the distribution of the NFT in the data market of the distributed storage 6 and the use of data resulting therefrom will be described.

In the Dfinity blockchain implemented by the distributed storage 6 or the blockchain system 5 accessible to the distributed storage 6, a search smart contract searching for the medical data is deployed. By the search smart contract, it is possible to execute a keyword search from the text and the like included in the medical data (the metadata) corresponding to each NFT. The data itself of the NFT imported in the blockchain system 5 may include a search keyword. In addition, each piece of medical data may include link data to a Web page in a Web server that is not illustrated, and the Web page may be searched. The Web page may include the thumbnail of the image included in the medical data.

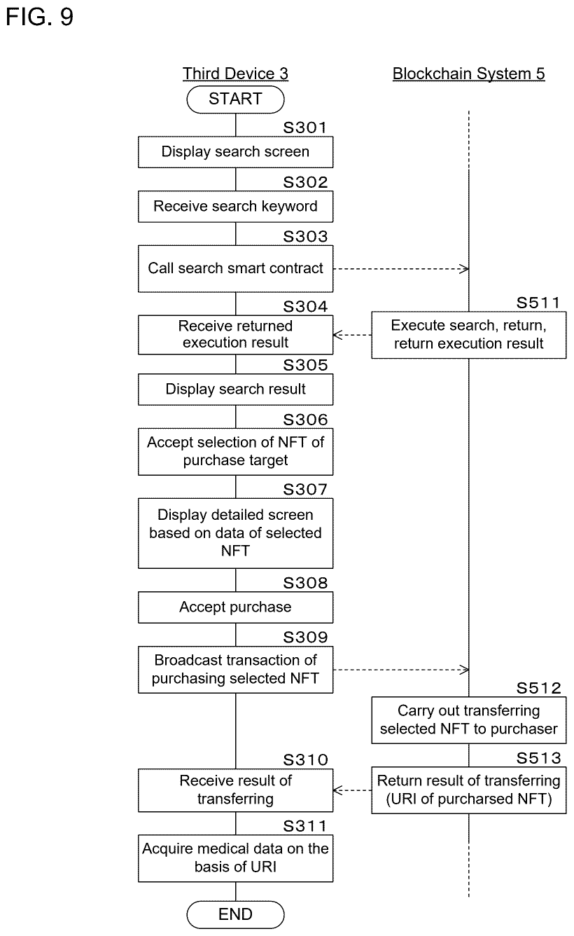

FIG. 9 is a sequence diagram illustrating transaction processing of the NFT of the medical data. The sequence in FIG. 9 is started by activating the third program P3 of the third device 3 to execute the search.

The processing unit 30 of the third device 3 displays a search screen, in accordance with the operation of the researcher or the like, on the basis of the third program P3 (step S301). The processing unit 30 accepts a search keyword on the search screen (step S302), and calls the search smart contract of the blockchain system 5 to execute the search (step S303). The search smart contract, for example, determines whether the outline of the medical data (the metadata) specified from the URI of each NFT includes the search keyword, and returns the result. The search smart contract that is called in step S303 may be implemented in the distributed storage 6. A search method is not limited to such a method.

The search smart contract returns an execution result of the search for the NFT stored in the distributed storage 6 to the third device 3 by the search keyword (step S511). The token ID of the NFT extracted by the search and the outline (the thumbnail of the Web page) of the medical data corresponding to the NFT are returned as the execution result.

The processing unit 30 of the third device 3 receives the returned execution result (step S304), and displays a search result on the display unit 33 (step S305). The processing unit 30 accepts the selection of an NFT of a purchase target from the search result (step S306). A detailed screen including the outline obtained by the data of the selected NFT (such as the token ID, the current owner, transaction history) and the URI, and a purchase button is displayed on the display unit 33 (step S307). In the detailed screen, a cost for purchasing the NFT and information required for the transaction are displayed.

In a case where the purchase is selected by the purchase button included in the detailed screen, the processing unit 30 accepts the selected purchase (step S308). The processing unit 30 broadcasts a transaction of purchasing the NFT of the purchase target to the blockchain system 5 (step S309).

In the blockchain system 5, processing of transferring the NFT of the purchase target to a purchaser is carried out (step S512), and the URI to the medical data included in the NFT that is a result of the processing is returned to the third device 3 (step S513).

In a case where the processing unit 30 of the third device 3 receives the result of the processing of transferring the NFT (step S310), the processing unit 30 acquires the medical data from the distributed storage 6, on the basis of the URI to the medical data included in the NFT (step S311), and ends the processing.

Accordingly, the researcher who is a third person is capable of acquiring the medical data by the NFT minted for the storage of the medical data. The medical data includes data diagnosed for the same user in a plurality of medical institutes in a cross-sectional manner, and is extremely valuable as a research target. The medical data includes data such as the “generation trust” and “authorization trust”, and by storing the data such that the data is difficult to be falsified, it is possible to verify that the data is retained without being falsified from the measurement device 4 ever since the data is generated.

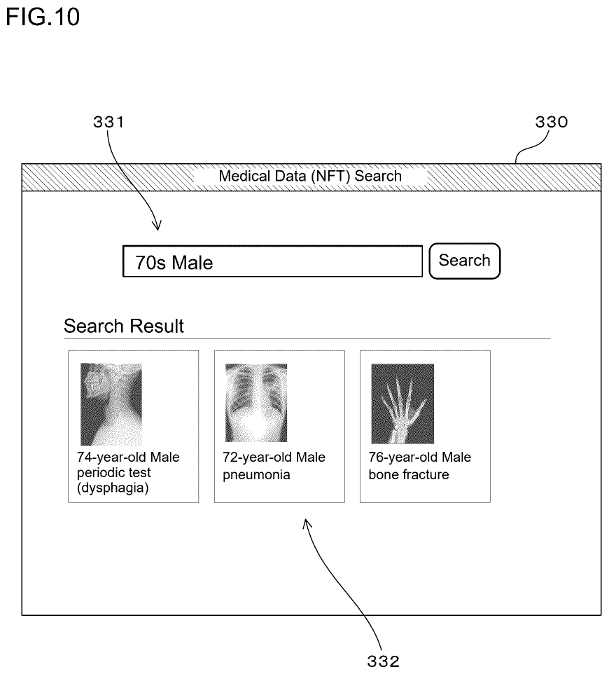

FIG. 10 illustrates a screen example displayed on the third device 3. FIG. 10 illustrates a search screen 330 displayed on the display unit 33 of the third device 3 in step S301. The search screen 330 includes a search section 331 for accepting the input of a keyword such as the age, the gender, and the name of diagnosed disease, and a display section 332 of a list of the outlines (the thumbnail in FIG. 10) of the medical data corresponding to each NFT of the search result. Each list displayed in the display section 332 is associated with a link to the detailed screen of each NFT, and in a case where the researcher operating the third device 3 selects any one by the operation unit 34, a transition to the detailed screen is performed.

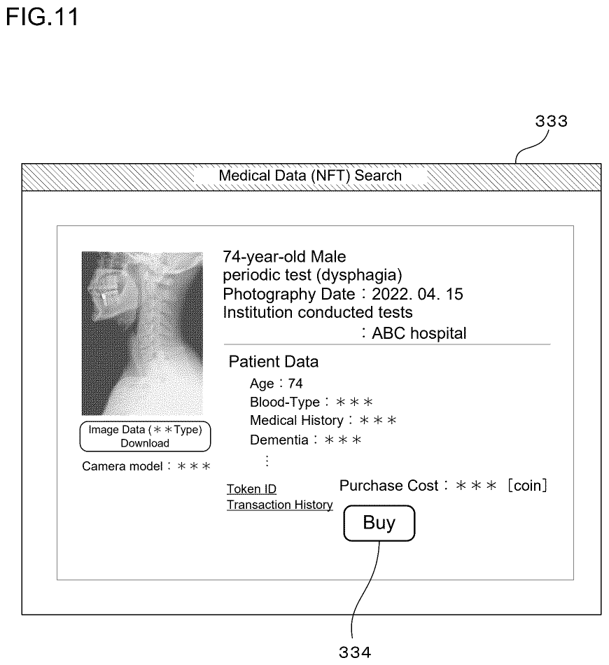

FIG. 11 illustrates a content example of a detailed screen 333. The detailed screen 333 includes the outline obtained by the URI of the selected NFT. In the detailed screen 333, the token ID of the selected NFT, and the individual identification data of the owner at the current time point or the name of the owner associated therewith are displayed. In the detailed screen 333, the attribute (such as the gender, the age, the blood type, the clinical history, and the nationality) of the patient of the medical data corresponding to the selected NFT is displayed. In the detailed screen 333, relevant data such as the transaction history of the selected NFT may be displayed.

The detailed screen 333 includes a purchase button 334. In a case where the purchase button 334 is selected from the operator of the third device 3, such as the researcher, the processing of transferring the NFT (S512) is executed.

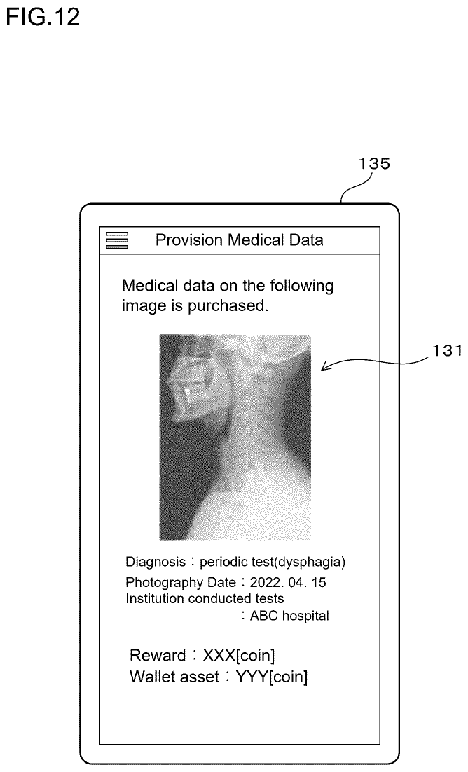

In a case where the processing of transferring the NFT is executed, it is preferable that such execution is notified to the original owner of the NFT. FIG. 12 illustrates a notification screen 135 with respect to the first device 1. A notification as a credit notification with respect to the wallet is transmitted to the first device 1, on the basis of the individual identification data of the original owner. In the notification screen 135, the outline of the contents of the sold NFT is displayed. In the notification screen 135 of FIG. 12, the image data 131 included in the medical data corresponding to the NFT is displayed as a representative. The notification screen 135 includes an asset of a reward for the patient associated with the transfer of the medical data, and the contents of the asset connected to the wallet. The asset required for the purchase may be legal currency, and various assets that can be distributed on the blockchain system 5 can be used, or any of the assets can be selected.

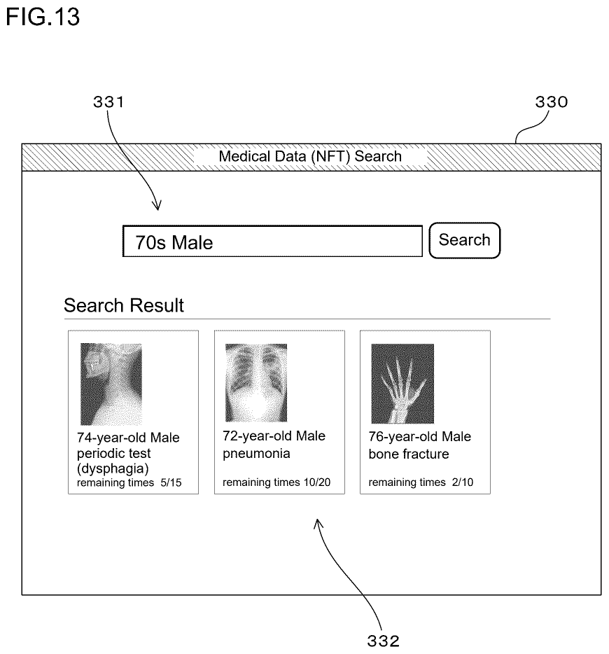

As described above, in the distributed storage 6, the medical data of the patient is stored, and the medical data can be used (downloaded) only from the third device 3 of a person who purchases the NFT. The once-purchased NFT can be secondarily distributed, and can also be transferred to another researcher. In addition, a plurality of NFTs can be minted for the original medical data, and can be purchased from a plurality of third devices 3. The asset is returned to the original owner each time when the plurality of NFTs are purchased. FIG. 13 illustrates the search screen 330 in a case where the plurality of NFTs are minted. In a case where the plurality of NFTs are minted, the number of times of minting and the number of remaining times of minting are displayed in the list of the search screen 330.

Note that it is preferable that the burning can be retroactively performed on the medical data that can be transacted in the data market in the distributed storage 6. Therefore, the medical data that is the metadata of the NFT includes a burning flag. The burning of the NFT can be executed on the basis of an offer of agreement withdrawal by the patient who is the original owner of the NFT or the agent thereof.

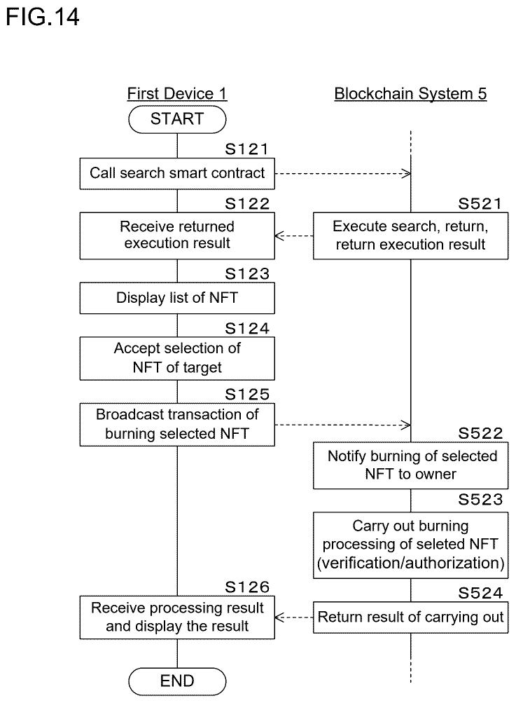

FIG. 14 is a sequence diagram illustrating the procedure of burning processing of the NFT. The processing procedure in FIG. 14 is started by the user or the assistant of the first device 1 activating the first program P1 of the first device 1.

The processing unit 10 calls the search smart contract of the blockchain system 5 to execute the search for the medical data of the user (the patient) of the first device 1 on the distributed storage 6 (step S121).

The search smart contract of the blockchain system 5 executes the NFT using the individual identification data of the user as the original owner from the NFT stored in the distributed storage 6, and returns the execution result of the search to the first device 1 (step S521). The token ID of the NFT extracted by the search and the outline (the thumbnail of the Web page) of the medical data corresponding to the NFT are returned as the execution result.

The processing unit 10 of the first device 1 receives the returned execution result (step S122), and displays the list of the NFTs of the medical data of the patient on the display unit 33 (step S123). The processing unit 10 accepts the selection of an NFT of a burning target from the list (step S124). In this case, an interface such as select all may be displayed.

The processing unit 10 broadcasts a transaction of burning the selected NFT to the blockchain system 5 (step S125).

In the blockchain system 5, processing of notifying the burning of the NFT to the owner of a target NFT is executed (step S522). In this case, a time limit may be provided until the next processing of step S523 is executed, or until the verification is accepted from each owner. For example, since the burning is performed within 10 days, the owner may be set to use the NFT within the available period.

In the blockchain system 5, the burning processing of the target NFT (the verification/authorization of the transaction) is carried out (step S523), and a processing result (success/failure) is returned to the first device 1 (step S524).

In the first device 1, the processing result is received (step S126), and the processing is ended.

As described above, even in a case where the NFT is purchased and the owner is changed, the burning of the NFT can be performed by the agreement withdrawal of the patient who is not the owner. Therefore, it is also possible to retroactively stop the distribution of the NFT, and stop the transaction of the data once distributed to the data market. Note that when the NFT is minted, a distribution time limit may be provided, and the burning may be automatically performed in a case where the time limit has passed.

In this embodiment, the remote medical examination system 100 is disclosed in which the NFT is minted for the data relevant to the medical care provided from the user who is the patient by using the NFT, and the data can be used in a cross-sectional manner. However, an information processing method in the present disclosure is not limited to the medical care, and can also be applied to the cross-sectional use of data relevant to agriculture, education, distribution, and the like.

For example, in the case of agriculture, cultivation data of a process from the planting to the harvesting of agricultural crops in a predetermined unit is stored in the distributed storage 6, and an NFT based on the cultivation data is minted. The cultivation data includes data such as the type of seed or seedling, the region of an agricultural land, weather information, a fertilizer, and a pesticide. By minting an NFT for cultivation data corresponding to an agricultural product that is harvested and shipped, it is possible to use the cultivation data in research such as the sharing of a cultivation method or the research of the growth situation of agricultural crops for each region. In addition, by minting the NFT, it is also possible to make the agricultural product produced by the cultivation data associated with the NFT valuable.

For example, in the case of education, learning data including an education facility, an instructional specialist, instructional contents, a test result, career information, and the like for each student is stored in the distributed storage 6, and an NFT based on the learning data is minted. The learning data can be used in education research based on the creation of statistical data of learning contents and career.

In addition, the information processing method can also be applied to distribution data until goods are distributed between a plurality of business operators and consumed, data relevant to events that are observed in different environments and can be the research target, and the like.

The embodiment disclosed as described above should be considered as illustrative in all respects and not restrictive. The scope of the present invention is indicated by the claims, and includes the meaning equivalent to the claims and all modifications within the scope.

### REFERENCE SIGNS LIST

100 remote medical examination system
1 first device
2 second device
3 third device
10, 20,30 processing unit
P1 first program
P2 second program
P3 third program
5 blockchain system
6 distributed storage

1-14. (canceled)

15. An information processing method for distribution medical data, comprising:
accepting, by a first device of a patient, agreement operation on providing of medical data relevant to a medical examination of the patient to a storage opened by any device;
accepting, by the first device, verification data for authenticity of the medical data;
transmitting the medical data for which the agreement and the verification can be accepted to a second device that is used in a medical institute of a diagnosis destination of the patient, with a measurement result by a measurement device measuring vitals of the patient, identification data of the measurement device, and reliability data indicating a degree of reliability of the measurement result;
storing, by the second device, the medical data for which the agreement and the verification can be accepted in the storage;
minting, by a smart contract of a blockchain system, a plurality of non-fungible tokens (NFTs) including an address for accessing the stored medical data for a blockchain account of the patient in the blockchain system, within a predetermined number of times of minting;
accepting, by the smart contract, a purchase of the NFT from a third device of another person, and
allowing, by the smart contract, an access to the medical data from a device of an NFT owner of the NFT if the device if it is successfully authenticated as being the device of the NFT owner.

16. The information processing method according to claim 15, wherein
the storage in which the medical data is stored is a blockchain.

17. The information processing method according to claim 15, wherein
the storage stores a plurality medical data, and
the smart contract of the blockchain system accepts a transaction of a purchase of an NFT corresponding to each of the plurality medical data.

18. The information processing method according to claim 15, wherein
a search smart contract of the blockchain system
accepts a search request for an NFT with a search key, and
returns a token ID of the NFT of which the search key is included in corresponding medical data, and a part of the medical data coincident with the search key.

19. The information processing method according to claim 18, wherein
a third device of a purchaser of the NFT accepts a purchase of an NFT selected from a search result, and
the smart contract of the blockchain system accepts a transaction of transferring the NFT from a patient corresponding to the NFT to the purchaser, from the third device.

20. The information processing method according to claim 17, wherein
in accordance with a transfer of the purchased NFT from a patient of the NFT to a blockchain account of a purchaser of the NFT, the smart contract of the blockchain system gives a part or entirety of a purchase cost to the blockchain account of the patient.

21. The information processing method according to claim 19, wherein
the smart contract of the blockchain system accepts a second purchase from an owner other than the patient who is an original owner of the NFT corresponding to each piece of medical data, and
the smart contract give a part of a purchase cost according to the second purchase to the blockchain account of the patient.

22. The information processing method according to claim 15, wherein
Even when not the owner of the NFT, the smart contract of the blockchain system accepts a transaction of burning the NFT from the blockchain account of the patient who is an original owner of the NFT.

23. The information processing method according to claim 15, wherein
in a case where the second device that is used in the medical institute receives the medical data for which the agreement and the verification can be accepted, the second device adds authorized data based on identification data of the medical institute.

24. The information processing method according to claim 15, wherein the measurement device outputs the measurement result with the reliability data including generation trust data indicating that data obtained from the measurement device is less likely to be falsified at a time point when generated.

25. The information processing method according to claim 15, wherein
the first device transmits the medical data with the reliability data including authorization trust data that is a result of verifying that the medical data is own data of the patient.

26. The information processing method according to claim 15, wherein
the first device transmits the medical data with the reliability data including cognition trust data indicating whether the patient of the medical data has cognitive ability.

27. The information processing system for distribution medical data, comprising:
a first device of a patient;
a second device that is used in a medical institute of a diagnosis destination of the patient; and
a blockchain system in which each of the patient and the medical institute has a blockchain account,
wherein
the first device accepts agreement operation on providing of medical data relevant to a medical examination of the patient to a storage opened by any device;
the first device accepts verification data for authenticity of the medical data;
the first device transmits the medical data for which the agreement and the verification can be accepted to a second device that is used in a medical institute of a diagnosis destination of the patient, with a measurement result by a measurement device measuring vitals of the patient, identification data of the measurement device, and reliability data indicating a degree of reliability of the measurement result;
the second device executes a process of storing the medical data for which the agreement and the verification can be accepted in the storage;
a smart contract of the blockchain system mints a plurality of non-fungible tokens (NFTs) including an address for accessing the stored medical data for a blockchain account of the patient in the blockchain system, within a predetermined number of times of minting;
the smart contract accepts a purchase of the NFT from a third device of another person, and
the smart contract allows an access to the medical data from a device of an NFT owner if the device if it is successfully authenticated as being the device of the NFT owner.

28. A non-transitory computer readable medium storing a program that cases a computer which is a node in a blockchain system, as a smart contract, to execute processing of:
the smart contract being called when an addition request for storing medical data relevant to a medical examination of a patient in a storage is broadcast,
minting a plurality of NFTs including an address for accessing the medical data on the storage including a measurement result by a measurement device measuring vitals of the patient, identification data of the measurement device, and reliability data indicating a degree of reliability of the measurement result to a blockchain account of the patient, within a predetermined number of times of minting;
accepting a purchase of the NFT from a third device of another person; and
allowing an access to the medical data from a device of an NFT owner.
